(19) United States
(12) Patent Application Publication
(10) Pub. No.: US 2024/0306693 A1
(43) Pub. Date: Sep. 19, 2024

(54) ENZYMATIC ENRICHMENT OF FOOD INGREDIENTS FOR SUGAR REDUCTION

(71) Applicant: BONUMOSE, INC., Carlottesville, VA (US)

(72) Inventors: Daniel Joseph WICHELECKI, Charlottesville, VA (US); Edwin O. ROGERS, Charlottesville, VA (US)

(21) Appl. No.: 18/263,830

(22) PCT Filed: Feb. 2, 2022
(86) PCT No.: PCT/US2022/014819

§ 371 Date: Aug. 1, 2023

Related U.S. Application Data
(60) Provisional application No. 63/144,815, filed on Feb. 2, 2021.

(51) Int. Cl. A23L 33/20; A21D 2/26; A23L 33/17; C12N 9/12; C12N 9/90; C12N 9/92

(52) U.S. Cl. CPC A23L 33/20; A21D 2/26; A23L 33/17; C12N 9/1205; C12N 9/90; C12N 9/92; C12Y 207/01144; C12Y 401/02022; C12Y 503/01009; C12Y 504/02002

(57) ABSTRACT

The invention relates to preparation of food ingredients enriched with a low-glycemic sugar replacement through enzymatic conversion. Food ingredients may be enriched with, for example, D-tagatose, D-allulose, D-allose, D-mannose, D-talose, and/or inositol by enzymatically converting saccharides found in flour, meal, ground tuber, ground pulse, ground bark, starch, malted grain or malt extract, maltodextrin, cellulose, cellodextrin, any of their derivatives (e.g., amylose, amylopectin, dextrin, cellobiose, etc.), and/or sucrose into D-tagatose, D-allulose, D-allose, D-mannose, D-talose and/or inositol. The enriched material can be used as a food ingredient instead of the low-glycemic sugar being purified for use as a food ingredient.

### CROSS-REFERENCE TO RELATED APPLICATIONS

This application claims priority to U.S. Provisional Application No. 63/144,815, filed on Feb. 2, 2021, the disclosure of which is incorporated by reference.

### FIELD OF THE INVENTION

The invention relates to preparation of food ingredients enriched with a low-glycemic sugar replacement through enzymatic conversion. More specifically, the invention relates to methods of preparing a food ingredient enriched with D-tagatose, D-allulose, D-allose, D-mannose, D-talose, and/or inositol by enzymatically converting saccharides found in flour, meal, ground tuber, ground pulse, ground bark, starch, malted grain or malt extract, maltodextrin, cellulose, cellodextrin, any of their derivatives (e.g., amylose, amylopectin, dextrin, cellobiose, etc.), and/or sucrose into D-tagatose, D-allulose, D-allose, D-mannose, D-talose and/or inositol where the enriched material is used as a food ingredient instead of the low-glycemic sugar being purified for use as a food ingredient.

### BACKGROUND

In order to fight diabetes and obesity, reduction of traditional sugar (including sucrose, glucose and other sugars) is a common goal among major food companies. With the advent of the mass production of low-glycemic sugar replacements (such as allulose or erythritol) this goal is being realized in more and more products. However, high costs of low-glycemic sugar replacements often result in the failure of economic feasibility for these healthier products in low-cost food stuffs. One method to reduce costs is to produce low-glycemic sugar replacements in an existing low-cost ingredient itself (such as flour, starch, or sucrose) rather than purifying and adding it as a separate ingredient. For example, currently allulose is produced predominantly through the enzymatic isomerization of fructose (WO 2014049373). Overall, the method suffers because of higher feedstock cost, the costly separation of allulose from fructose, and relatively low product yields.

There is a need to develop a cost-effective synthetic pathway for enrichment with low-glycemic sugars within food ingredient production where at least one step of the process involves an energetically favorable chemical reaction. Furthermore, there is a need for enrichment processes where the process steps of producing a low-glycemic sugar replacement can be conducted in one tank or bioreactor. There is also a need for a process of low-glycemic sugar replacement enrichment that can be conducted at a relatively low concentration of phosphate, where phosphate can be recycled, and/or the process does not require using adenosine triphosphate (ATP) as a source of phosphate. There is also a need for a low-glycemic sugar replacement enrichment pathway that does not require the use of the costly nicotinamide adenosine dinucleotide (NAD(H)) coenzyme in any of the reaction steps. There is also a need for enriching food ingredients with low-glycemic sugars without needing to first purify the food ingredient of non-starch components, when a lower conversion yield to low-glycemic sugars (relative to pure starch) is desirable due to the physical properties of the unconverted portion of the food ingredient and commercially acceptable to meet sugar reduction goals in food preparation.

### SUMMARY OF THE INVENTION

The inventions described herein relate to processes for preparing food ingredients enriched with low-glycemic sugar replacements utilizing enzymatic cascade reactions. The food ingredients applicable to this process are those which can produce glucose 1-phosphate without ATP (e.g., flour, meal, ground tuber, ground pulse, ground bark, starch, malted grain or malt extract, maltodextrin, cellulose, cellodextrin, their derivatives, and sucrose). In various aspects, the processes involve converting fructose 6-phosphate (F6P) to tagatose 6-phosphate (T6P), catalyzed by fructose 6-phosphate 4-epimerase (F6PE); and converting the T6P to tagatose, catalyzed by tagatose 6-phosphate phosphatase (T6PP). The inventions also relate to tagatose prepared by any of the processes described herein.

In various aspects, the processes involve converting fructose 6-phosphate (F6P) to psicose 6-phosphate (P6P), catalyzed by psicose 6-phosphate 3-epimerase (P6PE); and converting the P6P to allulose (also known as psicose), catalyzed by psicose 6-phosphate phosphatase (P6PP). The inventions also relate to allulose prepared by any of the processes described herein.

In various aspects, the processes involve converting fructose 6-phosphate (F6P) to psicose 6-phosphate (P6P), catalyzed by psicose 6-phosphate 3-epimerase (P6PE); converting the P6P to allose 6-phosphate (A6P), catalyzed by allose 6-phosphate isomerase (A6PI); and converting the A6P to allose, catalyzed by allose 6-phosphate phosphatase (A6PP). The inventions also relate to allose prepared by any of the processes described herein.

In various aspects, the processes involve converting fructose 6-phosphate (F6P) to mannose 6-phosphate (M6P), catalyzed by phosphomannose isomerase (PMI); and converting the M6P to mannose, catalyzed by mannose 6-phosphate phosphatase (M6PP). The inventions also relate to mannose prepared by any of the processes described herein.

In various aspects, the processes involve converting fructose 6-phosphate (F6P) to tagatose 6-phosphate (T6P), catalyzed by fructose 6-phosphate 4-epimerase (F6PE); converting the T6P to talose 6-phosphate (Tal6P), catalyzed by talose 6-phosphate isomerase (Tal6PI); and converting the Tal6P to talose, catalyzed by talose 6-phosphate phosphatase (Tal6PP). The inventions also relate to tagatose prepared by any of the processes described herein.

In various aspects, the processes involve converting glucose 6-phosphate (G6P) to inositol 3-phosphate (I3P), catalyzed by inositol synthase (IPS); and converting the I3P to inositol, catalyzed by inositol monophosphatase (IMP). The inventions also relate to inositol prepared by any of the processes described herein.

In some aspects of the invention, a process for preparing a low-glycemic sugar replacement also involves the step of converting glucose 6-phosphate (G6P) to the F6P, where the step is catalyzed by phosphoglucoisomerase (PGI). In other aspects, a process for low-glycemic sugar replacement synthesis also includes the step of converting glucose 1-phosphate (G1P) to the G6P, and this conversion step is catalyzed by phosphoglucomutase (PGM).

In various aspects, a process for preparing tagatose enrichment can involve converting a saccharide to the G1P, catalyzed by at least one enzyme; converting G1P to G6P, catalyzed by phosphoglucomutase (PGM); converting G6P to F6P, catalyzed by phosphoglucoisomerase (PGI); converting F6P to tagatose 6-phosphate (T6P), catalyzed by F6PE; and converting the T6P produced to tagatose, catalyzed by T6PP.

In various aspects, a process for preparing allulose enrichment can involve converting a saccharide to the G1P, catalyzed by at least one enzyme; converting G1P to G6P, catalyzed by phosphoglucomutase (PGM); converting G6P to F6P, catalyzed by phosphoglucoisomerase (PGI); converting F6P to psicose 6-phosphate (P6P), catalyzed by P6PE; and converting the P6P produced to allulose, catalyzed by P6PP.

In various aspects, a process for preparing allose enrichment can involve converting a saccharide to the G1P, catalyzed by at least one enzyme; converting G1P to G6P, catalyzed by phosphoglucomutase (PGM); converting G6P to F6P, catalyzed by phosphoglucoisomerase (PGI); converting F6P to psicose 6-phosphate (P6P), catalyzed by P6PE; converting P6P to allose 6-phosphate (A6P), catalyzed by A6PI, and converting the A6P produced to allose, catalyzed by A6PP.

In various aspects, a process for preparing mannose enrichment can involve converting a saccharide to the G1P, catalyzed by at least one enzyme; converting G1P to G6P, catalyzed by phosphoglucomutase (PGM); converting G6P to F6P, catalyzed by phosphoglucoisomerase (PGI); converting F6P to mannose 6-phosphate (M6P), catalyzed by PMI; and converting the M6P produced to mannose, catalyzed by M6PP.

In various aspects, a process for preparing inositol enrichment can involve converting a saccharide to the G1P, catalyzed by at least one enzyme; converting G1P to G6P, catalyzed by phosphoglucomutase (PGM); converting G6P to inositol 3-phosphate (I3P), catalyzed by IPS; and converting the I3P produced to inositol, catalyzed by IMP.

Processes of the invention are typically, but not necessarily, used to enrich low glycemic sugars in saccharide compositions that are used as food ingredients. For example, processes of the invention can be used to enrich low glycemic sugars in flour, meal, ground tuber, ground pulse, ground bark, starch, malted grain or malt extract, or derivatives of any of the foregoing substances.

Flour, which is generally defined as a powder made by grinding raw grains, roots, beans, nuts, or seeds. Flour or flour derivatives may contain one or more saccharides, including, but not limited to amylose, amylopectin, starch, soluble starch, amylodextrin, and maltodextrin. In some processes of the invention, low-glycemic sugar enrichment of a flour converts the flour to a flour derivative by enzymatic hydrolysis or by acid hydrolysis of starch. For example, a process of the invention may prepare a flour derivative by enzymatic hydrolysis of starch catalyzed by isoamylase, pullulanase, alpha-amylase, or a combination of two or more of these enzymes. Examples of flour derivatives prepared by a process of the invention include, but are not limited to amylose, amylopectin, soluble starch, amylodextrin, or maltodextrin. In another process of the invention, starch is converted to a starch derivative by enzymatic hydrolysis or by acid hydrolysis of starch. For example, a process of the invention may prepare a starch derivative by enzymatic hydrolysis of starch catalyzed by isoamylase, pullulanase, alpha-amylase, or a combination of two or more of these enzymes. A process for preparing low-glycemic sugar enrichment from a saccharide composition, including, for example, flour, starch, or their derivatives, may also add 4-glucan transferase (4GT).

### BRIEF DESCRIPTION OF THE FIGURES

FIG. 1A is a schematic diagram illustrating the enrichment of flour with a low-glycemic sugar, via partial conversion to maltodextrin, and enzymatic conversion of the maltodextrin to a low-glycemic sugar.

FIG. 1B is a schematic diagram illustrating the enrichment of a starch composition with a low-glycemic sugar, via partial conversion to maltodextrin, and enzymatic conversion of the maltodextrin to a low-glycemic sugar.

FIG. 1C is a schematic diagram illustrating the enrichment of a maltodextrin composition with a low-glycemic sugar, via enzymatic conversion of the maltodextrin to a low-glycemic sugar.

FIG. 1D is a schematic diagram illustrating the enrichment of a cellulose composition with a low-glycemic sugar, via partial conversion to cellodextrin, and enzymatic conversion of the cellodextrin to a low-glycemic sugar.

FIG. 1E is a schematic diagram illustrating the enrichment of a cellodextrin composition with a low-glycemic sugar, via enzymatic conversion of the cellodextrin to a low-glycemic sugar.

FIG. 1F is a schematic diagram illustrating the enrichment of a sucrose composition with a low-glycemic sugar, via partial conversion to fructose, and enzymatic conversion of the fructose to a low-glycemic sugar.

FIG. 2 is schematic diagram illustrating the enrichment of a starch composition with tagatose via partial conversion of the starch to maltodextrin, followed by steps performed without extracting the maltodextrin from the starch composition: converting the maltodextrin to glucose 1-phosphate (G1P); converting G1P to glucose 6-phosphate (G6P); converting G6P to fructose 6-phosphate (F6P); converting F6P to tagatose 6-phosphate (T6P); and converting the T6P to tagatose.

FIG. 3 is schematic diagram illustrating the enrichment of a starch composition with allulose via partial conversion of the starch to maltodextrin followed by steps performed without extracting the maltodextrin from the starch composition: converting the amylodextrin to glucose 1-phosphate (G1P); converting G1P to glucose 6-phosphate (G6P); converting G6P to fructose 6-phosphate (F6P); converting the F6P to psicose 6-phosphate (P6P); and converting the P6P to allulose (also known as psicose).

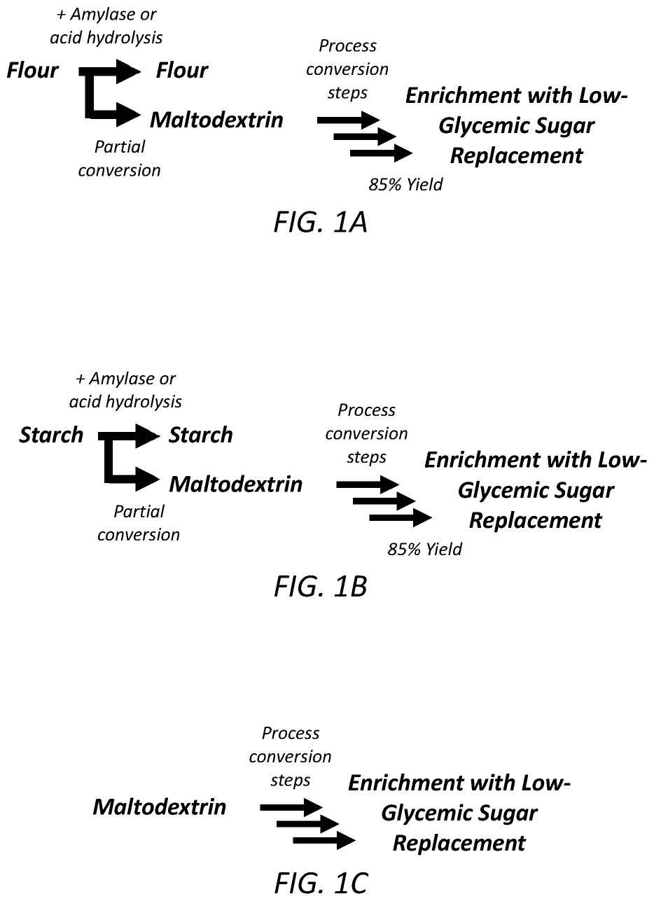

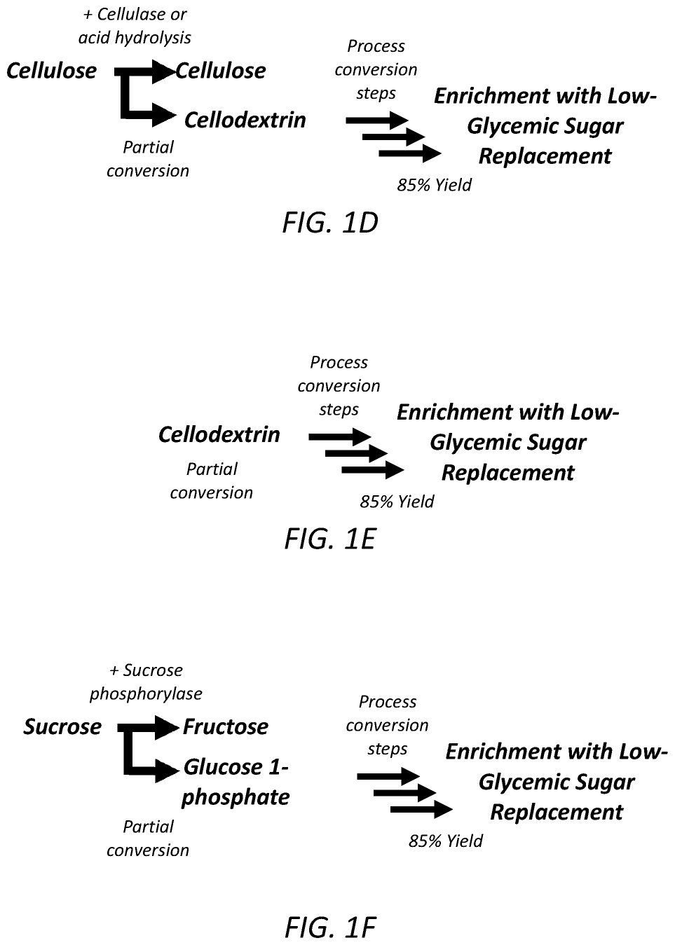

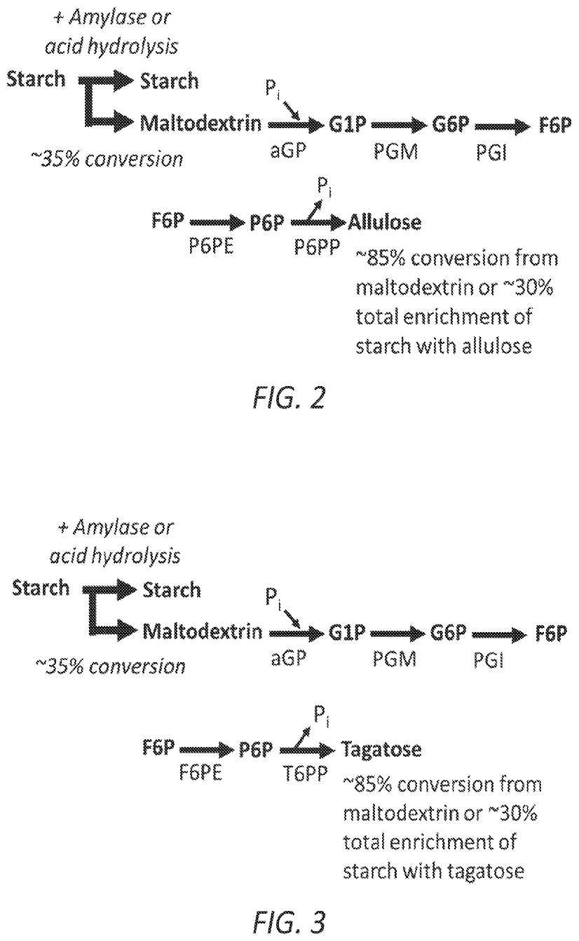

### DETAILED DESCRIPTION

The following description discloses the invention according to embodiments related to processes for producing a low-glycemic sugar-enriched saccharide composition from a saccharide composition which contains at least one saccharide. In general, a saccharide composition of the invention is a food ingredient, which can be used to produce glucose 1-phosphate without ATP, such as, for example, flour, meal, ground tuber, ground pulse, ground bark, starch, malted grain or malt extract, maltodextrin, cellulose, cellodextrin, their derivatives, and sucrose). In some processes of the invention, the saccharide composition is a flour, which may contain one or more of the following: starch, maltodextrin, sucrose, or cellulose. In some processes of the invention, the saccharide composition is a flour that is used as a food ingredient, including, for example, oat flour, potato flour, cassava flour, wheat flour, corn flour.

A process of the invention may include, but not necessarily require, steps of (i) converting a portion of the at least one saccharide and, optionally, at least one saccharide derivative to glucose 1-phosphate (G1P) using at least one enzyme; (ii) converting G1P to glucose 6-phosphate (G6P) using a phosphoglucomutase (PGM); and (iii) converting G6P to fructose 6-phosphate (F6P) using a phosphoglucoisomerase (PGI).

Further steps of a process of the invention, relating to the enrichment of particular low-glycemic sugars include:
(a) wherein the low-glycemic sugar-enriched saccharide composition is enriched for the low-glycemic sugar, tagatose,
   (1) converting fructose 6-phosphate (F6P) to tagatose 6-phosphate (T6P) using a fructose 6-phosphate epimerase (F6PE), and
   (2) converting the T6P produced to tagatose catalyzed by a tagatose 6-phosphate phosphatase (T6PP);
(b) wherein the low-glycemic sugar-enriched saccharide composition is enriched for the low-glycemic sugar, allulose,
   (1) converting fructose 6-phosphate (F6P) to psicose 6-phosphate (P6P) using a psicose 6-phosphate epimerase (P6PE), and
   (2) converting the P6P produced to allulose using a psicose 6-phosphate phosphatase (P6PP);
(c) wherein the low-glycemic sugar-enriched saccharide composition is enriched for the low-glycemic sugar, allose,
   (1) converting fructose 6-phosphate (F6P) to psicose 6-phosphate (P6P) using a psicose 6-phosphate 3-epimerase (P6PE),
   (2) converting the P6P to allose 6-phosphate (A6P) using an allose 6-phosphate isomerase (A6PI), and
   (3) converting the A6P to allose catalyzed by allose 6-phosphate phosphatase (A6PP);
(d) wherein the low-glycemic sugar-enriched saccharide composition is enriched for the low-glycemic sugar, mannose,
   (1) converting fructose 6-phosphate (F6P) to mannose 6-phosphate (M6P) using a phosphomannose isomerase (PMI) or phosphoglucose/phosphomannose isomerase (PGPMI), and
   (2) converting the M6P to mannose 6-phosphate (M6P) using a mannose 6-phosphate phosphatase (M6PP);
(e) wherein the low-glycemic sugar-enriched saccharide composition is enriched for the low-glycemic sugar, galactose,
   (1) converting fructose 6-phosphate (F6P) to tagatose 6-phosphate (T6P) using a fructose 6-phosphate epimerase (F6PE),
   (2) converting the T6P to galactose 6-phosphate (Gal6P) using a galactose 6-phosphate isomerase (Gal6PI), and
   (3) converting the Gal6P to galactose using a galactose 6-phosphate phosphatase (Gal6PP);
(f) wherein the low-glycemic sugar-enriched saccharide composition is enriched for the low-glycemic sugar, fructose,
   (1) converting fructose 6-phosphate (F6P) to fructose using a fructose 6-phosphate phosphatase (F6PP);
(g) wherein the low-glycemic sugar-enriched saccharide composition is enriched for the low-glycemic sugar, altrose,
   (1) converting fructose 6-phosphate (F6P) to psicose 6-phosphate (P6P) using a psicose 6-phosphate 3-epimerase (P6PE),
   (2) converting the P6P to altrose 6-phosphate (Alt6P) using an altrose 6-phosphate isomerase (Alt6PI), and
   (3) converting the Alt6P produced to altrose using an altrose 6-phosphate phosphatase (Alt6PP);
(h) wherein the low-glycemic sugar-enriched saccharide composition is enriched for the low-glycemic sugar, talose,
   (1) converting fructose 6-phosphate (F6P) to tagatose 6-phosphate (T6P) using a fructose 6-phosphate 4-epimerase (F6PE),
   (2) converting the T6P to talose 6-phosphate (Tal6P) using a talose 6-phosphate isomerase (Tal6PI), and
   (3) converting the Tal6P to talose using a talose 6-phosphate phosphatase (Tal6PP);
(i) wherein the low-glycemic sugar-enriched saccharide composition is enriched for the low-glycemic sugar, sorbose,
   (1) converting fructose 6-phosphate (F6P) to tagatose 6-phosphate (T6P) using a fructose 6-phosphate 4-epimerase (F6PE),
   (2) converting the T6P to sorbose 6-phosphate (S6P) using a sorbose 6-phosphate epimerase (S6PE), and
   (3) converting the S6P to sorbose using a sorbose 6-phosphate phosphatase (S6PP);
(j) wherein the low-glycemic sugar-enriched saccharide composition is enriched for the low-glycemic sugar, gulose,
   (1) converting fructose 6-phosphate (F6P) to tagatose 6-phosphate (T6P) using a fructose 6-phosphate 4-epimerase (F6PE),
   (2) converting the T6P to sorbose 6-phosphate (S6P) using a sorbose 6-phosphate epimerase (S6PE),
   (3) converting the S6P to gulose 6-phosphate (Gul6P) using a gulose 6-phosphate isomerase (Gul6PI), and
   (4) converting the Gul6P to gulose using a gulose 6-phosphate phosphatase (Gul6PP);
(k) wherein the low-glycemic sugar-enriched saccharide composition is enriched for the low-glycemic sugar, idose,
   (1) converting fructose 6-phosphate (F6P) to tagatose 6-phosphate (T6P) using a fructose 6-phosphate 4-epimerase (F6PE),
   (2) converting the T6P to sorbose 6-phosphate (S6P) using a sorbose 6-phosphate epimerase (S6PE),
   (3) converting the S6P to idose 6-phosphate (16P) using an idose 6-phosphate isomerase (16PI), and
   (4) converting the 16P to idose using an idose 6-phosphate phosphatase (16PP).

In another process of the invention, glucose 6-phosphate (G6P) is converted to inositol 3-phosphate (I3P), catalyzed by inositol synthase (IPS); and converting the I3P to inositol, catalyzed by inositol monophosphatase (IMP). The inventions also relate to inositol prepared by any of the processes described herein.

Details of the foregoing process steps are described in one or more of the following PCT publications, which are incorporated herein in their entireties, WO 2017/059278 (Tagatose), WO 2018/112139 (Allulose), WO 2018/169957 (Hexoses).

In certain processes of the invention, at least one saccharide is a starch, and a portion of the starch is converted to a starch derivative using acid hydrolysis or enzymatic hydrolysis. Enzymatic hydrolysis may be performed using, but not limited to, at least one of isoamylase, pullulanase, alpha-amylase.

In some processes of the invention, the saccharide derivative is maltodextrin, amylose, amylopectin, dextrin, cellodextrin, or cellobiose. In yet other processes of the invention, a saccharide composition may contain at least one saccharide, as well as at least one saccharide derivative, prior to implementing the process. In other words, the starting material may already contain one or more saccharides and one or more saccharide derivatives.

A process of the invention includes at least one energetically favorable chemical reaction. In some processes of the invention, the steps the process proceed as a cascade of energetically favorable chemical reactions, thereby permitting the process to be conducted in a single reaction vessel, e.g., a single bioreactor. Such processes of the invention may also be conducted under NAD(H)-free conditions.

Process steps of the invention are generally conducted at a temperature ranging from about 37° C. to about 160° C. Accordingly, in some processes of the invention, the process is, for example, conducted at a temperature of about 40° C., about 45° C., about 50° C., about 55° C., about 60° C., about 65° C., about 70° C., about 75° C., about 80° C., about 85° C., about 90° C., about 95° C., or about 100° C. In some processes of the invention, the reaction temperature is constant throughout the performance of the process.

Processes of the invention may be conducted under a range of pH conditions, generally ranging from 2.5-9.0. For example, a process of the invention may be conducted under pH conditions of 2.5 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.2, 6.2, 6.3, 6.4, 6.5, 6.6, 6.7, 6.8, 6.9, 7, 7.1, 7.2, 7.3, 7, 4, 7.5, 7.6, 7.7, 7.8, 7.9, 8, 8.1, 8.2, 8.3, 8.4, 8.5, 8.6, 8.7, 8.8, 8.0, or 9.

The reaction time of a process of the invention can be adjusted as necessary to obtain desired yields and/or accommodate other process objectives. Generally, a process of the invention is performed for about 1 hour to about 48 hours. Accordingly, in some processes of the invention, the process is performed for about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, or about 48 hours.

Phosphate ions (Pᵢ) produced by dephosphorylation of process intermediates may be recycled in one or more upstream process steps, such as, for example, for the step of converting a saccharide to G1P, when multiple or all process steps are conducted in a single bioreactor or reaction vessel. The ability to recycle phosphate in the disclosed processes allows for non-stoichiometric amounts of phosphate to be used, which keeps reaction phosphate concentrations low. This affects the overall pathway and the overall rate of the processes, but does not limit the activity of the individual enzymes and allows for overall efficiency of the low glycemic sugar making processes. Accordingly, the process steps in some methods of the invention are conducted without addition of exogenous phosphate, for example, in the absence of ATP (ATP-free) conditions, wherein phosphate ions produced by dephosphorylation steps of the process of the invention are recycled in upstream phosphorylation steps, and/or without the need for phosphate cofactors.

In processes of the invention, in which phosphate ions are added, the phosphate concentration is generally present at a concentration from about 0.1 mM to about 150 mM. For example, the phosphate concentration is about 0.1 mM, about 0.5 mM, about 1 mM, about 1.5 mM, about 2 mM, about 2.5 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 110 mM, about 120 mM, about 130 mM, about 140 mM, or about 150 mM.

A process of the invention for enriching a low-glycemic sugar in a saccharide composition may convert about 0.1% to about 100% of the saccharides in the saccharide composition. For example, a process of the invention may convert 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, or 100% of the saccharides in flour (e.g., oat flour, potato flour, cassava flour, wheat flour, corn flour, soy flour, rice flour, pea flour, banana flour, or plantain flour), meal, ground tuber, ground pulse, ground bark, starch, malted grain or malt extract, cellulose, and/or sucrose to a saccharide derivative, such as, for example, maltodextrin, cellobiose, cellodextrin, and/or their derivatives. Subsequently, in the same or different processes of the invention, the process further converts about 0.1% to about 95% of the saccharide derivative a to a low-glycemic sugar. For example, about 0.1%, 0.5%, 1%, 5%, 10%, 15%, 20%, 25%, 30%, 35%, 40%, 45%, 50%, 55%, 60%, 65%, 70%, 75%, 80%, 85%, 90%, or 95% of the maltodextrin yielded by the conversion of a saccharide in a saccharide composition in a process of the invention is converted to tagatose, allulose, mannose, talose, inositol, and/or allose.

### Examples

The following examples exemplify processes of the claimed invention.

### Materials and Methods to be Utilized in the Examples

Chemicals. All chemicals, including corn starch, soluble starch, maltodextrins, maltose, glucose, filter paper were reagent grade or higher and purchased from Sigma-Aldrich (St. Louis, MO, USA) or Fisher Scientific (Pittsburgh, PA, USA), unless otherwise noted. Restriction enzymes, T4 ligase, and Phusion DNA polymerase were purchased from New England Biolabs (Ipswich, MA, USA). Oligonucleotides were synthesized either by Integrated DNA Technologies (Coralville, IA, USA) or Eurofins MWG Operon (Huntsville, AL, USA). Regenerated amorphous cellulose used in enzyme purification was prepared from Avicel PH105 (FMC BioPolymer, Philadelphia, PA, USA) through its dissolution and regeneration, as described in: Ye et al., Fusion of a family 9 cellulose-binding module improves catalytic potential of Clostridium thermocellum cellodextrin phosphorylase on insoluble cellulose. Appl. Microbiol. Biotechnol. 2011; 92:551-560. Escherichia coli Sig10 (Sigma-Aldrich, St. Louis, MO, USA) was used as a host cell for DNA manipulation and E. coli BL21 (DE3) (Sigma-Aldrich, St. Louis, MO, USA) was used as a host cell for recombinant protein expression. ZYM-5052 media including either 100 mg L⁻¹ ampicillin or 50 mg L⁻¹ kanamycin was used for E. coli cell growth and recombinant protein expression. Cellulase from Trichoderma reesei (Catalog number: C2730) and pullulanase (Catalog number: P1067) were purchased from Sigma-Aldrich (St. Louis, MO, USA) and produced by Novozymes (Franklinton, NC, USA).

Production and purification of recombinant enzymes. The E. coli BL21 (DE3) strain harboring a protein expression plasmid was incubated in a 1-L Erlenmeyer flask with 100 mL of ZYM-5052 media containing either 100 mg L⁻¹ ampicillin or 50 mg L⁻¹ kanamycin. Cells were grown at 30° C. with rotary shaking at 220 rpm for 16-24 hours. The cells were harvested by centrifugation at 12° C. and washed once with either 20 mM HEPES (pH 7.5) containing 50 mM NaCl and 5 mM MgCl₂ (heat precipitation and cellulose-binding module) or 20 mM HEPES (pH 7.5) containing 300 mM NaCl and 5 mM imidazole (Ni purification). The cell pellets were re-suspended in the same buffer and lysed by ultra-sonication (Fisher Scientific Sonic Dismembrator Model 500; 5 s pulse on and 10 s off, total 21 min at 50% amplitude). After centrifugation, the target proteins in the supernatants were purified.

Three approaches were used to purify the various recombinant proteins. His-tagged proteins were purified by the Profinity IMAC Ni-Charged Resin (Bio-Rad, Hercules, CA, USA). Fusion proteins containing a cellulose-binding module (CBM) and self-cleavage intein were purified through high-affinity adsorption on a large surface-area regenerated amorphous cellulose. Heat precipitation at 70-95° C. for 5-30 min was used to purify hyperthermostable enzymes. The purity of the recombinant proteins was examined by sodium dodecyl sulfate polyacrylamide gel electrophoresis (SDS-PAGE).

Enzymes used and their activity assays. Alpha-glucan phosphorylase (αGP) from Thermus sp. CCB_US3_UF1 (Uniprot G8NCC0) was used. Activity was assayed in 50 mM sodium phosphate buffer (pH 7.2) containing 1 mM MgCl₂, and 30 mM maltodextrin at 50° C. The reaction was stopped via filtration of enzyme with a Vivaspin® 2 concentrator (10,000 MWCO) (Vivaproducts, Inc., Littleton, MA, USA). Glucose 1-phosphate (G1P) was measured using a glucose hexokinase/G6PDH assay kit (Sigma Aldrich, Catalog No. GAHK20-1KT) supplemented with 25 U/mL phosphoglucomutase. A unit (U) is described as μmol/min.

Phosphoglucomutase (PGM) from Caldibacillus debilis (Uniprot A0A150LLZ1) was used. Activity was measured in 50 mM HEPES buffer (pH 7.2) containing 5 mM MgCl₂ and 5 mM G1P at 50° C. The reaction was stopped via filtration of enzyme with a Vivaspin® 2 concentrator (10,000 MWCO). The product glucose 6-phosphate (G6P) was determined using a hexokinase/G6PDH assay kit (Sigma Aldrich, Catalog No. GAHK20-1KT).

Phosphoglucoisomerase (PGI) from Thermus thermophilus (Uniprot ID Q5SLL6) was used. Activity was measured in 50 mM HEPES buffer (pH 7.2) containing 5 mM MgCl₂ and 10 mM F6P at 50° C. The reaction was stopped via filtration of enzyme with a Vivaspin® 2 concentrator (10,000 MWCO). The product glucose 6-phosphate (G6P) was determined using a hexokinase/G6PDH assay kit (Sigma Aldrich, Catalog No. GAHK20-1KT).

The recombinant isoamylase from Sulfolobus tokodaii is described in Cheng et al., Doubling power output of starch biobattery treated by the most thermostable isoamylase from an archaeon Sulfolobus tokodaii. Scientific Reports 2015; 5:13184. Its activities were assayed as described.

The recombinant 4-alpha-glucanotransferase (4GT) from Thermococcus litoralis is described in Jeon et al. 4-α-Glucanotransferase from the Hyperthermophilic Archaeon Thermococcus Litoralis. Eur. J. Biochem. 1997; 248:171-178. Its activity was measured as described. The 4GT from Anaerolinea thermophila strain DSM 14523 (Uniprot E8MXP8) was used and assayed as described in Jeon et al.

Sucrose phosphorylase from Thermoanaerobacterium xylanolyticum (strain ATCC 49914/DSM 7097/LX-11) was used. Its activity was measured in 50 mM HEPES buffer (pH 7.5) containing 10 mM sucrose and 12 mM organic phosphate. Glucose 1-phosphate (G1P) was measured using a glucose hexokinase/G6PDH assay kit supplemented with 25 U/mL phosphoglucomutase as with alpha-glucan phosphorylase.

Fructose 6-phosphate epimerase (F6PE) from Thermanaerothrix daxensis (Uniprot A0A0P6XN50) was used. Activity was measured in 50 mM HEPES buffer (pH 7.2) containing 5 mM MgCl₂, 0.5 mM MnCl₂, and 10 mM F6P at 50° C. The product, tagatose 6-phosphate (T6P), was quantified by using excess tagatose 6-phosphate phosphatase to produce tagatose. The reactions were stopped via filtration of enzymes using a Vivaspin® 2 concentrator (10,000 MWCO) and analyzed via HPLC (Agilent 1100 series), using an Agilent Hi-Plex® H-column and refractive index detector. The sample was run in 5 mM H₂SO₄ at 0.6 mL/min for 15.5 minutes at 65° C. and tagatose was quantified using tagatose standards.

Tagatose 6-phosphate phosphatase (T6PP) from Sphaerobacter thermophilus strain DSM 20745 (Uniprot ID D1C7G9) was used. Activity was measured in 50 mM HEPES buffer (pH 7.2) containing 5 mM MgCl₂ and 10 mM T6P at 50° C. The reaction was stopped via filtration of enzyme with a Vivaspin® 2 concentrator (10,000 MWCO). Tagatose production was determined as described for F6PE.

Psicose 6-phosphate epimerase (P6PE) from Thermoanaerobacterium thermosaccharolyticum (Uniprot A0A223HZI7) was used. Activity was measured in 50 mM HEPES buffer (pH 7.2) containing 5 mM MgCl₂, 0.5 mM CoCl₂, and 10 mM F6P at 50° C. The product, psicose 6-phosphate (P6P), was quantified by using excess psicose 6-phosphate phosphatase to produce psicose. The reactions were stopped via filtration of enzymes using a Vivaspin® 2 concentrator (10,000 MWCO) and analyzed via HPLC (Agilent 1100 series), using an Agilent Hi-Plex® H-column and refractive index detector. The sample was run in 5 mM H₂SO₄ at 0.6 mL/min for 15.5 minutes at 65° C. and psicose was quantified using psicose standards.

Psicose 6-phosphate phosphatase (P6PP) from Methanosarcina thermophila CHTI-55 (Uniprot ID A0A0E3NCH4) was used. Activity was measured in 50 mM HEPES buffer (pH 7.2) containing 5 mM MgCl₂, 0.5 mM CoCl₂, excess P6PE, and 10 mM F6P at 50° C. The reaction was stopped via filtration of enzyme with a Vivaspin® 2 concentrator (10,000 MWCO). Psicose production was determined as described for P6PE.

Allose 6-phosphate isomerase (A6PI) from Heliobacterium modesticaldum (Uniprot BOTI66) was used. Activity was measured in 50 mM HEPES buffer (pH 7.2) containing 5 mM MgCl₂, 0.5 mM CoCl₂, excess P6PE, and 10 mM F6P at 50° C. The product, allose 6-phosphate (A6P), was quantified by using excess allose 6-phosphate phosphatase to produce allose. The reactions were stopped via filtration of enzymes using a Vivaspin® 2 concentrator (10,000 MWCO) and analyzed via HPLC (Agilent 1100 series), using an Agilent Hi-Plex® H-column and refractive index detector. The sample was run in 5 mM H₂SO₄ at 0.6 mL/min for 15.5 minutes at 65° C. and allose was quantified using allose standards.

Allose 6-phosphate phosphatase (A6PP) from Thermotoga maritima (Uniprot ID Q9X0Y1) was used. Activity was measured in 50 mM HEPES buffer (pH 7.2) containing 5 mM MgCl₂, 0.5 mM CoCl₂, excess P6PE, excess A6PI, and 10 mM F6P at 50° C. The reaction was stopped via filtration of enzyme with a Vivaspin® 2 concentrator (10,000 MWCO). Allose production was determined as described for A6PI.

Phosphomannose isomerase (PMI) from Caldithrix abyssi DSM 13497 (Uniprot H1XQS6) was used. Activity was measured in 50 mM HEPES buffer (pH 7.2) containing 5 mM MgCl₂ and 10 mM F6P at 50° C. The product, mannose 6-phosphate (M6P), was quantified by using excess mannose 6-phosphate phosphatase to produce mannose. The reactions were stopped via filtration of enzymes using a Vivaspin® 2 concentrator (10,000 MWCO) and analyzed via HPLC (Agilent 1100 series), using an Agilent Hi-Plex® H-column and refractive index detector. The sample was run in 5 mM H₂SO₄ at 0.6 mL/min for 15.5 minutes at 65° C. and mannose was quantified using mannose standards.

Mannose 6-phosphate phosphatase (M6PP) from Thermobifida cellulosilytica TB100 (Uniprot ID A0A147KII8) was used. Activity was measured in 50 mM HEPES buffer (pH 7.2) containing 5 mM MgCl₂, excess PMI, and 10 mM F6P at 50° C. The reaction was stopped via filtration of enzyme with a Vivaspin® 2 concentrator (10,000 MWCO). Mannose production was determined as described for PMI.

Talose 6-phosphate isomerase (Tal6PI) is used. Activity is measured in 50 mM HEPES buffer (pH 7.2) containing 5 mM MgCl₂, 0.5 mM MnCl₂, excess F6PE, and 10 mM F6P at 50° C. The product, talose 6-phosphate (Tal6P), is quantified by using excess talose 6-phosphate phosphatase to produce talose. The reactions are stopped via filtration of enzymes using a Vivaspin® 2 concentrator (10,000 MWCO) and analyzed via HPLC (Agilent 1100 series), using an Agilent Hi-Plex® H-column and refractive index detector. The sample is run in 5 mM H₂SO₄ at 0.6 mL/min for 15.5 minutes at 65° C. and talose is quantified using talose standards.

Talose 6-phosphate phosphatase (T6PP) is used. Activity is measured in 50 mM HEPES buffer (pH 7.2) containing 5 mM MgCl₂, excess F6PE, excess Tal6PI, and 10 mM F6P at 50° C. The reaction is stopped via filtration of enzyme with a Vivaspin® 2 concentrator (10,000 MWCO). Talose production is determined as described for Tal6PI.

Inositol-3-phosphate synthase from Archaeoglobus fulgidus (Uniprot 028480) was used (Chen et al. Inositol-3-phosphate synthase from Archaeoglobus fulgidus is a Class II Aldolase. Biochemistry 2000; 39:12415-12423). Its activity was measured as described therein.

Inositol Monophosphatase from Thermotoga maritima (Uniprot 033832) was used (Stec et al. MJ0109 is an enzyme that is both an inositol monophosphatase and the ‘missing’ archaeal fructose-1,6-bisphosphatase. Nat. Struct. Biol. 7:1046-1050(2000)). Its activity was measured as described therein.

Enzyme units used in each Example below can be increased or decreased to adjust the reaction time as desired. For example, if one wanted to perform an enrichment in 2 h instead of 8 h, the units of the enzymes could theoretically be increased about 4-fold. Conversely, if the enrichment process is performed in 24 h instead of 8 h, the enzyme units could theoretically be decreased about 3-fold.

Similarly, the enzyme units, or reaction time, in each Example below can be adjusted for the degree of enrichment one desires. For example, if 85% enrichment is seen in a 24-hour reaction with 5 U total enzyme one can either decrease the reaction time to 8.5 hours or enzyme amount to 1.75 U to achieve 30% enrichment.

Example 1. To validate enrichment of tagatose in maltodextrin, a 0.20 mL reaction mixture containing 20 g/L maltodextrin, 50 mM phosphate buffered saline pH 7.2, 5 mM MgCl₂, 0.05 mM MnCl₂, 0.05 U of αGP, 0.05 U PGM, 0.05 U PGI, 0.05 U F6PE, and 0.05 U T6PP was incubated at 50° C. for 24 hours. The reaction was stopped via filtration of enzyme with a Vivaspin® 2 concentrator (10,000 MWCO). Tagatose enrichment was detected and quantified using an Agilent 1100 series HPLC with refractive index detector and an Agilent Hi-Plex H-column. The mobile phase was 5 mM H₂SO₄, which ran at 0.6 mL/min and 65° C. An enrichment of 9.2 g/L tagatose was obtained. Standards of various concentrations of tagatose were used to quantify the enrichment.

Example 2. To validate enhanced enrichment of tagatose in maltodextrin, a reaction mixture containing 200 g/L maltodextrin, 10 mM acetate buffer (pH 5.5), 5 mM MgCl₂, and 0.1 g/L isoamylase was incubated at 80° C. for 24 hours. This was used to create another reaction mixture containing 20 g/L isoamylase treated maltodextrin, 50 mM phosphate buffered saline pH 7.2, 5 mM MgCl₂, 0.05 mM MnCl₂, 0.05 U of αGP, 0.05 U PGM, 0.05 U PGI, 0.05 U F6PE, and 0.05 U T6PP was incubated at 50° C. for 24 hours. Production of tagatose was quantified as in Example 1. The enrichment of tagatose was increased to 16 g/L with the pretreatment of maltodextrin by isoamylase.

Example 3. To further increase enrichment of tagatose in maltodextrin, 0.05 U 4-glucan transferase (4GT) was added to the reaction described in Example 2. A 0.2 mL reaction mixture containing 20 g/L isoamylase treated maltodextrin (see example 2), 50 mM phosphate buffered saline pH 7.2, 5 mM MgCl₂, 0.05 mM MnCl₂, 0.05 U of αGP, 0.05 U PGM, 0.05 U PGI, 0.05 U F6PE, 0.05 U T6PP, and 0.05 U 4GT was incubated at 50° C. for 24 hours. Enrichment with tagatose was quantified as in Example 1. The enrichment of tagatose was increased to 17.7 g/L with the addition of 4GT to IA-treated maltodextrin.

Example 4. To enrich for tagatose in sucrose, a reaction mixture containing 10 g/L sucrose, 50 mM phosphate buffered saline pH 7.2, 5 mM MgCl₂, 0.05 mM MnCl₂, 0.15 U sucrose phosphorylase, 0.15 PGM, 0.15 U PGI, 0.15 U F6PE, and 0.15 U T6PP was incubated at 50° C. for 8 hours. Enrichment with tagatose was verified as in Example 1.

Example 5. To validate enrichment of tagatose in potato flour, a reaction mixture containing 200 g/L potato flour, 0.02 g/L CaCl₂, and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material was enriched for tagatose as described in Example 3.

Example 6. To validate enrichment of tagatose in cassava flour, a reaction mixture containing 200 g/L cassava flour, 0.02 g/L CaCl₂, and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. at 95° C. at 95° C. before neutralization with 500 mM NaOH. This process converts the starch within the flour to maltodextrin for enrichment. The resulting material was enriched for tagatose as described in Example 3.

Example 7. To validate enrichment of tagatose in potato starch, a reaction mixture containing 200 g/L potato starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material was enriched for tagatose as described in Example 3.

Example 8. To validate enrichment of tagatose in cassava starch, a reaction mixture containing 200 g/L cassava starch, 0.02 g/L CaCl₂, and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material was enriched for tagatose as described in Example 3.

Example 9. To validate enrichment of tagatose in pea starch, a reaction mixture containing 200 g/L pea starch, 0.02 g/L CaCl₂, and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material was enriched for tagatose as described in Example 3.

Example 10. To validate enrichment of tagatose in corn starch, a reaction mixture containing 200 g/L pea starch, 0.02 g/L CaCl₂), and 2 μL 1:100 diluted Liquozyme® Supra 2.2× is incubated at 95° C. for 14 min (mix ever 2 min after 8 min). At 14 min, the pH is adjusted between 3 and 3.5 with 20% HCl and held for 24 min at 95° C. before neutralization with 500 mM NaOH. This process produces maltodextrin from starch. The resulting material is enriched for tagatose as described in Example 3.

Example 11. To validate enrichment of allulose in maltodextrin, a 0.2 mL reaction mixture containing 20 g/L isoamylase treated maltodextrin (see example 2), 50 mM phosphate buffered saline pH 7.2, 5 mM MgCl₂, 0.05 mM CoCl₂, 0.05 U of αGP, 0.05 U PGM, 0.05 U PGI, 0.05 U P6PE, 0.05 U P6PP, and 0.05 U 4GT was incubated at 50° C. for 24 hours. Enrichment with allulose was detected and quantified using an Agilent 1100 series HPLC with refractive index detector and an SUPELCOGEL Pb column. The mobile phase was ultrapure water, which ran at 0.6 mL/min and 80° C. Standards of various concentrations of allulose were used to quantify the yield. The enrichment of allulose in maltodextrin was 18.8 g/L with the addition of 4GT to IA-treated maltodextrin.

Example 12. To enrich for allulose in sucrose, a reaction mixture containing 10 g/L sucrose, 50 mM phosphate buffered saline pH 7.2, 5 mM MgCl₂, 0.05 mM CoCl₂, 0.15 U sucrose phosphorylase, 0.15 PGM, 0.15 U PGI, 0.15 U P6PE, and 0.15 U P6PP was incubated at 50° C. for 8 hours. Enrichment with allulose was verified as in Example 11.

Example 13. To validate enrichment of allulose in potato flour, a reaction mixture containing 200 g/L potato flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for allulose as described in Example 11.

Example 14. To validate enrichment of allulose in cassava flour, a reaction mixture containing 200 g/L cassava flour, 0.02 g/L CaCl₂, and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for allulose as described in Example 11.

Example 15. To validate enrichment of allulose in potato starch, a reaction mixture containing 200 g/L potato starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for allulose as described in Example 11.

Example 16. To validate enrichment of allulose in cassava starch, a reaction mixture containing 200 g/L cassava starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for allulose as described in Example 11.

Example 17. To validate enrichment of allulose in pea starch, a reaction mixture containing 200 g/L pea starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for allulose as described in Example 11.

Example 18. To validate enrichment of allulose in corn starch, a reaction mixture containing 200 g/L pea starch, 0.02 g/L CaCl₂), and 2 μL 1:100 diluted Liquozyme® Supra 2.2× is incubated at 95° C. for 14 min (mix ever 2 min after 8 min). At 14 min, the pH is adjusted between 3 and 3.5 with 20% HCl and held for 24 min at 95° C. before neutralization with 500 mM NaOH. This process produces maltodextrin from starch. The resulting material is enriched for allulose as described in Example 11.

Example 19. To validate enrichment of allose in maltodextrin, a 0.2 mL reaction mixture containing 20 g/L isoamylase treated maltodextrin (see example 2), 50 mM phosphate buffered saline pH 7.2, 5 mM MgCl₂, 0.05 mM CoCl₂, 0.05 U of αGP, 0.05 U PGM, 0.05 U PGI, 0.05 U P6PE, 0.05 U A6PI, 0.05 U A6PP, and 0.05 U 4GT was incubated at 50° C. for 24 hours. Enrichment with allulose was verified using an Agilent 1100 series HPLC with refractive index detector and an SUPELCOGEL Pb column. The mobile phase was ultrapure water, which ran at 0.6 mL/min and 80° C. Standards of various concentrations of allose were used as verification.

Example 20. To enrich for allose in sucrose, a reaction mixture containing 10 g/L sucrose, 50 mM phosphate buffered saline pH 7.2, 5 mM MgCl₂, 0.05 mM CoCl₂, 0.15 U sucrose phosphorylase, 0.15 PGM, 0.15 U PGI, 0.15 U P6PE, 0.15 U A6PI, and 0.15 U A6PP is incubated at 50° C. for 8 hours. Enrichment with allose is verified as in Example 19.

Example 21. To validate enrichment of allose in potato flour, a reaction mixture containing 200 g/L potato flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for allose as described in Example 19.

Example 22. To validate enrichment of allose in cassava flour, a reaction mixture containing 200 g/L cassava flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for allose as described in Example 19.

Example 23. To validate enrichment of allose in potato starch, a reaction mixture containing 200 g/L potato starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for allose as described in Example 19.

Example 24. To validate enrichment of allose in cassava starch, a reaction mixture containing 200 g/L cassava starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for allose as described in Example 19.

Example 25. To validate enrichment of allose in pea starch, a reaction mixture containing 200 g/L pea starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for allose as described in Example 19.

Example 26. To validate enrichment of allose in corn starch, a reaction mixture containing 200 g/L pea starch, 0.02 g/L CaCl₂), and 2 μL 1:100 diluted Liquozyme® Supra 2.2× is incubated at 95° C. for 14 min (mix ever 2 min after 8 min). At 14 min, the pH is adjusted between 3 and 3.5 with 20% HCl and held for 24 min at 95° C. before neutralization with 500 mM NaOH. This process produces maltodextrin from starch. The resulting material is enriched for allose as described in Example 19.

Example 27. To validate enrichment of mannose in maltodextrin, a 0.2 mL reaction mixture containing 20 g/L isoamylase treated maltodextrin (see example 2), 50 mM phosphate buffered saline pH 7.2, 5 mM MgCl₂, 0.05 U of αGP, 0.05 U PGM, 0.05 U PGI, 0.05 U PMI, 0.05 U M6PP, and 0.05 U 4GT was incubated at 50° C. for 24 hours. Enrichment with mannose was verified using an Agilent® 1100 series HPLC with refractive index detector and an SUPELCOGEL Pb column. The mobile phase was ultrapure water, which ran at 0.6 mL/min and 80° C. Standards of various concentrations of mannose were used as verification. The enrichment of mannose in maltodextrin was 17.6 g/L with the addition of 4GT to IA-treated maltodextrin.

Example 28. To enrich for mannose in sucrose, a reaction mixture containing 10 g/L sucrose, 50 mM phosphate buffered saline pH 7.2, 5 mM MgCl₂, 0.15 U sucrose phosphorylase, 0.15 PGM, 0.15 U PGI, 0.15 U PMI, and 0.15 U M6PP was incubated at 50° C. for 8 hours. Enrichment with mannose was verified as in Example 27.

Example 29. To validate enrichment of mannose in potato flour, a reaction mixture containing 200 g/L potato flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for mannose as described in Example 27.

Example 30. To validate enrichment of mannose in cassava flour, a reaction mixture containing 200 g/L cassava flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for mannose as described in Example 27.

Example 31. To validate enrichment of mannose in potato starch, a reaction mixture containing 200 g/L potato starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for mannose as described in Example 27.

Example 32. To validate enrichment of mannose in cassava starch, a reaction mixture containing 200 g/L cassava starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for mannose as described in Example 27.

Example 33. To validate enrichment of mannose in pea starch, a reaction mixture containing 200 g/L pea starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for mannose as described in Example 27.

Example 34. To validate enrichment of mannose in corn starch, a reaction mixture containing 200 g/L pea starch, 0.02 g/L CaCl₂), and 2 μL 1:100 diluted Liquozyme® Supra 2.2× is incubated at 95° C. for 14 min (mix ever 2 min after 8 min). At 14 min, the pH is adjusted between 3 and 3.5 with 20% HCl and held for 24 min at 95° C. before neutralization with 500 mM NaOH. This process produces maltodextrin from starch. The resulting material is enriched for mannose as described in Example 27.

Example 35. To validate enrichment of talose in maltodextrin, a 0.2 mL reaction mixture containing 20 g/L isoamylase treated maltodextrin (see example 2), 50 mM phosphate buffered saline pH 7.2, 5 mM MgCl₂, 0.5 mM MnCl₂, 0.05 U of αGP, 0.05 U PGM, 0.05 U PGI, 0.05 U F6PE, 0.05 Tal6PI, 0.05 U Tal6PP, and 0.05 U 4GT is incubated at 50° C. for 24 hours. Enrichment with talose is verified using an Agilent® 1100 series HPLC with refractive index detector and an SUPELCOGEL Pb column. The mobile phase is ultrapure water, which runs at 0.6 mL/min and 80° C. Standards of various concentrations of talose are used as verification.

Example 36. To enrich for talose in sucrose, a reaction mixture containing 10 g/L sucrose, 50 mM phosphate buffered saline pH 7.2, 5 mM MgCl₂, 0.05 mM MnCl₂, 0.15 U sucrose phosphorylase, 0.15 PGM, 0.15 U PGI, 0.15 U F6PE, 0.15 U Tal6PI, and 0.15 U Tal6PP is incubated at 50° C. for 8 hours. Enrichment with talose is verified as in Example 35.

Example 37. To validate enrichment of talose in potato flour, a reaction mixture containing 200 g/L potato flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for talose as described in Example 35.

Example 38. To validate enrichment of talose in cassava flour, a reaction mixture containing 200 g/L cassava flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for talose as described in Example 35.

Example 39. To validate enrichment of talose in potato starch, a reaction mixture containing 200 g/L potato starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for talose as described in Example 35.

Example 40. To validate enrichment of talose in cassava starch, a reaction mixture containing 200 g/L cassava starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for talose as described in Example 35.

Example 41. To validate enrichment of talose in pea starch, a reaction mixture containing 200 g/L pea starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for talose as described in Example 35.

Example 42. To validate enrichment of talose in corn starch, a reaction mixture containing 200 g/L pea starch, 0.02 g/L CaCl₂), and 2 μL 1:100 diluted Liquozyme® Supra 2.2× is incubated at 95° C. for 14 min (mix ever 2 min after 8 min). At 14 min, the pH is adjusted between 3 and 3.5 with 20% HCl and held for 24 min at 95° C. before neutralization with 500 mM NaOH. This process produces maltodextrin from starch. The resulting material is enriched for talose as described in Example 35.

Example 43. The enzymes for enriching maltodextrin with inositol were alpha-glucan phosphorylase from T. maritima (KEGG CDS, TM0769), phosphoglucomutase from Thermococcus kodakaraensis (KEGG CDS, TK1108), inositol 3-phosphatase from Archaeoglobus fulgidus (KEGG CDS, AF1794), and inositol monophosphatase from T. maritima (KEGG CDS, TM1415). In a 0.75-mL reaction mixture with a 100-mM HEPES buffer (pH 7.2) containing 10 g/L maltodextrin, 10 mM phosphate, 5 mM MgCl₂ and 0.5 mM ZnCl₂, 0.05 U/ml of αGP, 1 U/mL PGM, 0.05 U/mL IPS, and 2 U/mL of IMP were added. The reaction was conducted at 60° C. for 40 hours. Enrichment with inositol was verified using an Agilent 1100 series HPLC with refractive index detector and an Aminex HPX-87C column. The mobile phase is ultrapure water, which runs at 0.6 mL/min and 80° C. Standards of various concentrations of inositol were used as verification. An enrichment of 3.6 g/L inositol was found.

Example 44. Maltodextrin contains many branches that impede αGP action. Isoamylase was used as in Example 2, yielding linear amylodextrin. Isoamylase-pretreated starch resulted in a higher inositol enrichment of 6.0 g/L in the final product, in comparison with 3.6 g/L of inositol using a maltodextrin that has not been pretreated using isoamylase.

Example 45. To further increase inositol enrichment on debranched maltodextrin, 4-alpha-glucanotransferase was used, which can rearrange two short maltodextrins (e.g., maltose and maltotriose) to a longer maltodextrin and glucose. The addition of 0.05 U/ml 4-alpha-glucanotransferase to Example 44 further increased inositol enrichment to 7.0 g/L.

Example 46. To test another method for enhancement of inositol enrichment in maltodextrin, pullulanase was used to debranch maltodextrin first for 10 hours at 37° C.; and then 1 U/mL αGP, 1 U/mL PGM, 2 U/mL IPS and 2 U/mL IMP were added. After 40 hours at 60° C., the final inositol concentration was 7.3 g/L.

Example 47. To enrich for inositol in sucrose, a reaction mixture containing 10 g/L sucrose, 50 mM phosphate buffered saline pH 7.2, 5 mM MgCl₂, 0.15 U sucrose phosphorylase, 0.15 PGM, 0.15 U IPS, and 0.15 U IMP is incubated at 50° C. for 8 hours. Enrichment with inositol is verified as in Example 43.

Example 45. To validate enrichment of inositol in potato flour, a reaction mixture containing 200 g/L potato flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for inositol as described in Example 43.

Example 46. To validate enrichment of inositol in cassava flour, a reaction mixture containing 200 g/L cassava flour, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process converts the starch within the flour to maltodextrin for enrichment. The resulting material is enriched for inositol as described in Example 43.

Example 47. To validate enrichment of inositol in potato starch, a reaction mixture containing 200 g/L potato starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for inositol as described in Example 43.

Example 48. To validate enrichment of inositol in cassava starch, a reaction mixture containing 200 g/L cassava starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for inositol as described in Example 43.

Example 49. To validate enrichment of inositol in pea starch, a reaction mixture containing 200 g/L pea starch, 0.02 g/L CaCl₂), and 2 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material is enriched for inositol as described in Example 43.

Example 50. To validate enrichment of inositol in corn starch, a reaction mixture containing 200 g/L pea starch, 0.02 g/L CaCl₂), and 2 μL 1:100 diluted Liquozyme® Supra 2.2× is incubated at 95° C. for 14 min (mix ever 2 min after 8 min). At 14 min, the pH is adjusted between 3 and 3.5 with 20% HCl and held for 24 min at 95° C. before neutralization with 500 mM NaOH. This process produces maltodextrin from starch. The resulting material is enriched for inositol as described in Example 43.

Example 51. F6P and its precursor G6P are feed stocks for low-glycemic sugar enrichment in cellulose. To test for F6P production from Avicel (commercial cellulose), Sigma cellulase was used to hydrolyze cellulose at 50° C. To remove beta-glucosidase from commercial cellulase, 10 filter paper units/ml of cellulase was mixed to 10 g/L Avicel® at an ice-water bath for 10 min. After centrifugation at 4° C., the supernatant containing beta-glucosidase was decanted. Avicel® that was bound with cellulase containing endoglucanase and cellobiohydrolase was resuspended in a citrate buffer (pH 4.8) for hydrolysis at 50° C. for three days. The cellulose hydrolysate was mixed with 5 U/mL cellodextrin phosphorylase, 5 U/L cellobiose phosphorylase, 5 U/ml of αGP, 5 U/mL PGM, and 5 U/mL PGI in a 100 mM HEPES buffer (pH 7.2) containing 10 mM phosphate, 5 mM MgCl₂ and 0.5 mM ZnCl₂. The reaction was conducted at 60° C. for 72 hours and high concentrations of F6P were found (small amounts of glucose and no cellobiose). F6P was detected using a fructose 6-phosphate kinase (F6PK)/pyruvate dehydrogenase (PK)/lactate dehydrogenase (LD) coupled enzyme assay where a decrease in absorbance at 340 nm indicates production of F6P. A 200 μL reaction was prepared that contained 50 mM HEPES (pH 7.2), 5 mM MgCl₂, 10 mM G6P, 1.5 mM ATP, 1.5 mM phosphoenol pyruvate, 200 μM NADH, 0.1 U PGI, 5 U PK, and 5 U LD. The decrease in 340 nm is converted to the concentration of F6P using Beer's Law and the extinction coefficient of NADH. Glucose was detected using a hexokinase/G6PDH assay kit (Sigma Aldrich, Catalog No. GAHK20-1KT).

Example 52. To increase F6P yields from Avicel, Avicel was pretreated with concentrated phosphoric acid to produce amorphous cellulose (RAC), as described in Zhang et al. A transition from cellulose swelling to cellulose dissolution by o-phosphoric acid: evidence from enzymatic hydrolysis and supramolecular structure. Biomacromolecules 2006; 7:644-648. To remove beta-glucosidase from commercial cellulase, 10 filter paper units/mL of cellulase was mixed with 10 g/L RAC in an ice-water bath for 5 min. After centrifugation at 4° C., the supernatant containing beta-glucosidase was decanted. The RAC that was bound with cellulase containing endoglucanase and cellobiohydrolase was resuspended in a citrate buffer (pH 4.8) for hydrolysis at 50° C. for 12 hours. The RAC hydrolysate was mixed with 5 U/mL cellodextrin phosphorylase, 5 U/L cellobiose phosphorylase, 5 U/ml of αGP, 5 U/mL PGM, and 5 U/mL PGI in a 100 mM HEPES buffer (pH 7.2) containing 10 mM phosphate, 5 mM MgCl₂ and 0.5 mM ZnCl₂. The reaction was conducted at 60° C. for 72 hours. High concentrations of F6P and glucose were recovered because no enzymes were added to convert glucose to F6P. F6P was detected using the coupled enzyme assay described above. Glucose was detected using a hexokinase/G6PDH assay kit (Sigma Aldrich, Catalog No. GAHK20-1KT).

Example 53. To further increase F6P yields from RAC, polyphosphate glucokinase and polyphosphate were added. To remove beta-glucosidase from commercial cellulase, 10 filter paper units/mL of cellulase was mixed with 10 g/L RAC in an ice-water bath for 5 min. After centrifugation at 4° C., the supernatant containing beta-glucosidase was decanted. The RAC that was bound with cellulase containing endoglucanase and cellobiohydrolase was re-suspended in a citrate buffer (pH 4.8) for hydrolysis at 50° C. was incubated in a citrate buffer (pH 4.8) for hydrolysis at 50° C. for 12 hours. The RAC hydrolysate was mixed with 5 U/mL polyphosphate glucokinase, 5 U/mL cellodextrin phosphorylase, 5 U/mL cellobiose phosphorylase, 5 U/ml of αGP, 5 U/mL PGM, and 5 U/mL PGI in a 100 mM HEPES buffer (pH 7.2) containing 50 mM polyphosphate, 10 mM phosphate, 5 mM MgCl₂ and 0.5 mM ZnCl₂. The reaction was conducted at 50° C. for 72 hours. F6P was found in high concentrations with only small amounts of glucose now present. F6P was detected using the coupled enzyme assay described above. Glucose was detected using a hexokinase/G6PDH assay kit (Sigma Aldrich, Catalog No. GAHK20-1KT).

Example 54. To validate tagatose production from F6P (and therefore enrichment feasibility from cellulose), 2 g/L F6P was mixed with 1 U/ml fructose 6-phosphate epimerase (F6PE) and 1 U/ml tagatose 6-phosphate phosphatase (T6PP) in 50 mM HEPES buffer (pH 7.2) containing 5 mM MgCl₂ and 0.5 mM MnCl₂. The reaction was incubated for 16 hours at 50° C.˜ 100% conversion of F6P to tagatose was seen via HPLC (Agilent 1100 series) using an Agilent Hi-Plex H-column and refractive index detector. The sample was run in 5 mM H₂SO₄ at 0.6 mL/min at 65° C. and conversion verified using tagatose standards.

Example 55. To validate allulose production from F6P (and therefore enrichment feasibility from cellulose), 2 g/L F6P was mixed with 1 U/ml P6PE and 1 U/ml P6PP in 50 mM HEPES buffer (pH 7.2) containing 5 mM MgCl₂ and 0.5 mM CoCl₂. The reaction was incubated for 16 hours at 50° C.˜ 100% conversion of F6P to allulose was seen via HPLC (Agilent 1100 series) using an Agilent Hi-Plex H-column and refractive index detector. The sample was run in 5 mM H₂SO₄ at 0.6 mL/min at 65° C. and conversion verified using allulose standards.

Example 56. To validate allose production from F6P (and therefore enrichment feasibility from cellulose), 2 g/L F6P was mixed with 1 U/ml P6PE, 1 U/ml A6PI, and 1 U/ml A6PP in 50 mM HEPES buffer (pH 7.2) containing 5 mM MgCl₂ and 0.5 mM CoCl₂. The reaction was incubated for 16 hours at 50° C. Conversion of F6P to allose was seen via HPLC (Agilent 1100 series) using an Agilent Hi-Plex H-column and refractive index detector. The sample was run in 5 mM H₂SO₄ at 0.6 mL/min at 65° C. and conversion verified using allulose standards.

Example 57. To validate mannose production from F6P (and therefore enrichment feasibility from cellulose), 2 g/L F6P was mixed with 1 U/ml PMI and 1 U/ml M6PP in 50 mM HEPES buffer (pH 7.2) containing 5 mM MgCl₂. The reaction was incubated for 16 hours at 50° C.˜ 100% conversion of F6P to mannose was seen via HPLC (Agilent 1100 series) using an Agilent Hi-Plex® H-column and refractive index detector. The sample was run in 5 mM H₂SO₄ at 0.6 mL/min at 65° C. and conversion verified using mannose standards.

Example 58. To validate talose production from F6P (and therefore enrichment feasibility from cellulose), 2 g/L F6P is mixed with 1 U/ml F6PE, 1 U/ml Tal6PI, and 1 U/ml Tal6PP in 50 mM HEPES buffer (pH 7.2) containing 5 mM MgCl₂ and 0.5 mM MnCl₂. The reaction is incubated for 16 hours at 50° C.˜ 100% conversion of Tal6P to talose is seen via HPLC (Agilent 1100 series) using an Agilent Hi-Plex® H-column and refractive index detector. The sample is run in 5 mM H₂SO₄ at 0.6 mL/min at 65° C. and is verified using talose standards.

Example 59. To validate inositol production from G6P (F6P precursor; and therefore enrichment feasibility from cellulose), 2 g/L G6P was mixed with 1 U/ml IPS and 1 U/ml IMP in 50 mM HEPES buffer (pH 7.2) containing 5 mM MgCl₂. The reaction was incubated for 16 hours at 50° C.˜ 100% conversion of F6P to inositol was seen via HPLC (Agilent 1100 series) using an Agilent Hi-Plex H-column and refractive index detector. The sample was run in 5 mM H₂SO₄ at 0.6 mL/min at 65° C. and was verified using inositol standards.

Example 60. A 0.20 mL reaction mixture containing 10 g/L maltodextrin, 50 mM phosphate buffered saline pH 7.2, 5 mM MgCl₂, 0.01 U of αGP, 0.01 U PGM, and 0.01 U PGI was incubated at 50° C. for 24 hours. The reaction was stopped via filtration of enzyme with a Vivaspin® 2 concentrator (10,000 MWCO). The product, fructose 6-phosphate (F6P), was determined using a fructose 6-phosphate kinase (F6PK)/pyruvate dehydrogenase (PK)/lactate dehydrogenase (LD) coupled enzyme assay where a decrease in absorbance at 340 nm indicates production of F6P as described above. The final concentration of F6P (reaction must go through G6P to reach F6P with current enzymes) after 24 hours was 3.6 g/L.

Example 61. To validate enrichment of tagatose in oat flour, a reaction mixture containing 200 g/L oat flour, 0.02 g/L CaCl₂), and 4 μL 1:100 diluted Liquozyme® Supra 2.2× (amylase) was incubated at 95° C. for 14 min (mix ever 3 min after 8 min). At 14 min, the pH was adjusted between 3 and 3.5 with 20% HCl and held for 24 min at 95° C. before neutralization with 500 mM NaOH. This process produces maltodextrin from starch while deactivating amylase. The resulting material was enriched for tagatose. A reaction containing 200 g/L debranched (see Example 2) and amylase treated oat flour, 25 mM phosphate buffered saline pH 7.2, 5 mM MgCl₂, 0.5 mM MnCl₂, 0.5 U of αGP, 0.5 U PGM, 0.5 U PGI, 0.5 U F6PE, 0.5 U T6PP, and 0.5 U 4GT was incubated at 50° C. for 24 hours. Enrichment with tagatose was detected and quantified using an Agilent 1100 series HPLC with refractive index detector and an SUPELCOGEL® Pb column. The mobile phase was ultrapure water, which ran at 0.6 mL/min and 80° C. Standards of various concentrations of tagatose were used to quantify the yield. 60.5 g/L of tagatose was produced from this source material.

Example 62. To validate enhanced enrichment of tagatose in barley malt extract, a reaction mixture containing 205.1 g/L debranched malt extract (see Example 2), 25 mM phosphate buffered saline pH 7.2, 5 mM MgCl₂, 0.5 mM MnCl₂, 0.125 U of αGP, 0.125 U PGM, 0.125 U PGI, 0.125 U F6PE, 0.125 U T6PP, and 0.125 U 4GT was incubated at 50° C. for 24 hours. Production of tagatose was quantified as in Example 1.52 g/L of tagatose was produced from this source material.

Example 63. To validate enrichment of allulose in oat flour, a reaction mixture containing 200 g/L oat flour, 0.02 g/L CaCl₂), and 4 μL 1:100 diluted Liquozyme® Supra 2.2× was incubated at 95° C. for 14 min (mix ever 2 min after 8 min). At 14 min, the pH was adjusted between 3 and 3.5 with 20% HCl and held for 24 min at 95° C. before neutralization with 500 mM NaOH. This process produces maltodextrin from starch. The resulting material was enriched for allulose. A reaction containing 50 g/L debranched (see Example 2) and amylase treated oat flour, 25 mM phosphate buffered saline pH 7.2, 5 mM MgCl₂, 0.5 mM CoCl₂, 0.125 U of αGP, 0.125 U PGM, 0.125 U PGI, 0.125 U P6PE, 0.125 U P6PP, and 0.125 U 4GT was incubated at 50° C. for 24 hours. Enrichment with allulose was detected and quantified as in Example 11. 17.5 g/L of allulose was produced from this source material.

Example 64. To validate enhanced enrichment of allulose in barley malt extract, a reaction mixture containing 205.1 g/L debranched malt extract (see Example 2), 25 mM phosphate buffered saline pH 7.2, 5 mM MgCl₂, 0.5 mM CoCl₂, 0.125 U of αGP, 0.125 U PGM, 0.125 U PGI, 0.125 U P6PE, and 0.125 U P6PP was incubated at 50° C. for 24 hours. Enrichment with allulose was detected and quantified as in Example 11. 10.4 g/L of allulose was produced from this source material.

Example 65. To further increase enrichment of allulose in barley malt extract, 0.125 U 4-glucan transferase (4GT) was added to the reaction described in Example 64. Enrichment with allulose was detected and quantified as in Example 11. 51.4 g/L of allulose was produced from this source material.

1. A process for producing a low-glycemic sugar-enriched saccharide composition from a saccharide composition comprising at least one saccharide,
the process comprising:
(i) converting a portion of the total amount of the at least one saccharide in the saccharide composition to a saccharide derivative;
(ii) converting the saccharide derivative to glucose 1-phosphate (G1P) using at least one enzyme;
(iii) converting G1P to glucose 6-phosphate (G6P) using a phosphoglucomutase (PGM);
(iv) converting G6P to fructose 6-phosphate (F6P) using a phosphoglucoisomerase (PGI); and
(a) wherein the low-glycemic sugar-enriched saccharide composition is enriched for the low-glycemic sugar, tagatose,
(1) converting fructose 6-phosphate (F6P) to tagatose 6-phosphate (T6P) using a fructose 6-phosphate epimerase (F6PE), and
(2) converting the T6P produced to tagatose catalyzed by a tagatose 6-phosphate phosphatase (T6PP);
(b) wherein the low-glycemic sugar-enriched saccharide composition is enriched for the low-glycemic sugar, allulose,
(1) converting fructose 6-phosphate (F6P) to psicose 6-phosphate (P6P) using a psicose 6-phosphate epimerase (P6PE), and
(2) converting the P6P produced to allulose using a psicose 6-phosphate phosphatase (P6PP);
(c) wherein the low-glycemic sugar-enriched saccharide composition is enriched for the low-glycemic sugar, allose,
(1) converting fructose 6-phosphate (F6P) to psicose 6-phosphate (P6P) using a psicose 6-phosphate 3-epimerase (P6PE),
(2) converting the P6P to allose 6-phosphate (A6P) using an allose 6-phosphate isomerase (A6PI), and
(3) converting the A6P to allose catalyzed by allose 6-phosphate phosphatase (A6PP);
(d) wherein the low-glycemic sugar-enriched saccharide composition is enriched for the low-glycemic sugar, mannose,
(1) converting fructose 6-phosphate (F6P) to mannose 6-phosphate (M6P) using a phosphomannose isomerase (PMI) or phosphoglucose/phosphomannose isomerase (PGPMI), and
(2) converting the M6P to mannose 6-phosphate (M6P) using a mannose 6-phosphate phosphatase (M6PP);
(e) wherein the low-glycemic sugar-enriched saccharide composition is enriched for the low-glycemic sugar, galactose,
(1) converting fructose 6-phosphate (F6P) to tagatose 6-phosphate (T6P) using a fructose 6-phosphate epimerase (F6PE),
(2) converting the T6P to galactose 6-phosphate (Gal6P) using a galactose 6-phosphate isomerase (Gal6PI), and
(3) converting the Gal6P to galactose using a galactose 6-phosphate phosphatase (Gal6PP);
(f) wherein the low-glycemic sugar-enriched saccharide composition is enriched for the low-glycemic sugar, fructose,
(1) converting fructose 6-phosphate (F6P) to fructose using a fructose 6-phosphate phosphatase (F6PP);
(g) wherein the low-glycemic sugar-enriched saccharide composition is enriched for the low-glycemic sugar, altrose,
(1) converting fructose 6-phosphate (F6P) to psicose 6-phosphate (P6P) using a psicose 6-phosphate 3-epimerase (P6PE),
(2) converting the P6P to altrose 6-phosphate (Alt6P) using an altrose 6-phosphate isomerase (Alt6PI), and
(3) converting the Alt6P produced to altrose using an altrose 6-phosphate phosphatase (Alt6PP);
(h) wherein the low-glycemic sugar-enriched saccharide composition is enriched for the low-glycemic sugar, talose,
(1) converting fructose 6-phosphate (F6P) to tagatose 6-phosphate (T6P) using a fructose 6-phosphate 4-epimerase (F6PE),
(2) converting the T6P to talose 6-phosphate (Tal6P) using a talose 6-phosphate isomerase (Tal6PI), and
(3) converting the Tal6P to talose using a talose 6-phosphate phosphatase (Tal6PP);
(i) wherein the low-glycemic sugar-enriched saccharide composition is enriched for the low-glycemic sugar, sorbose,
(1) converting fructose 6-phosphate (F6P) to tagatose 6-phosphate (T6P) using a fructose 6-phosphate 4-epimerase (F6PE),
(2) converting the T6P to sorbose 6-phosphate (S6P) using a sorbose 6-phosphate epimerase (S6PE), and
(3) converting the S6P to sorbose using a sorbose 6-phosphate phosphatase (S6PP);
(j) wherein the low-glycemic sugar-enriched saccharide composition is enriched for the low-glycemic sugar, gulose,
(1) converting fructose 6-phosphate (F6P) to tagatose 6-phosphate (T6P) using a fructose 6-phosphate 4-epimerase (F6PE),
(2) converting the T6P to sorbose 6-phosphate (S6P) using a sorbose 6-phosphate epimerase (S6PE),
(3) converting the S6P to gulose 6-phosphate (Gul6P) using a gulose 6-phosphate isomerase (Gul6PI), and
(4) converting the Gul6P to gulose using a gulose 6-phosphate phosphatase (Gul6PP);
(k) wherein the low-glycemic sugar-enriched saccharide composition is enriched for the low-glycemic sugar, idose,
(1) converting fructose 6-phosphate (F6P) to tagatose 6-phosphate (T6P) using a fructose 6-phosphate 4-epimerase (F6PE),
(2) converting the T6P to sorbose 6-phosphate (S6P) using a sorbose 6-phosphate epimerase (S6PE),
(3) converting the S6P to idose 6-phosphate (16P) using an idose 6-phosphate isomerase (16PI), and
(4) converting the 16P to idose using an idose 6-phosphate phosphatase (16PP).

2. The process of claim 1, wherein either acid hydrolysis or enzymatic hydrolysis is used to convert the at least one saccharide to a saccharide derivative.

3. The process of claim 2, wherein the enzymatic hydrolysis is performed using at least one of isoamylase, pullulanase, alpha-amylase, and cellulase.

4. The process of claim 1, wherein the at least one saccharide derivative is maltodextrin, amylose, amylopectin, dextrin, cellodextrin, cellulose, cellobiose.

5. The process of claim 1, wherein the at least one saccharide is starch, maltodextrin, sucrose, or cellulose.

6. The process of claim 1, wherein the at least one enzyme in step 1(ii), converting the saccharide derivative to G1P, is alpha-glucan phosphorylase (αGP), sucrose phosphorylase, cellodextrin phosphorylase, or cellobiose phosphorylase.

7. The process of claim 1, wherein the process steps are conducted at a temperature ranging from about 40° C. to about 160° C., and/or at a pH ranging from about 3.0 to about 8.0.

8. The process of claim 1, wherein the process steps are conducted ATP-free, NAD(H)-free, at a phosphate concentration from about 0 mM to about 150 mM, the phosphate is recycled, and/or at least one step of the process involves an energetically favorable chemical reaction.

9. The process of claim 1, wherein 4-glucan transferase (4GT) is added to the process.

10. The process of claim 1, wherein the process steps are conducted in one bioreactor or in a plurality of bioreactors arranged in series.

11. The process of claim 1, wherein the saccharide composition is a flour, meal, ground tuber, ground pulse, or ground bark comprising starch.

12. The process of claim 11, wherein the flour is oat flour, potato flour, cassava flour, wheat flour, corn flour, soy flour, rice flour, pea flour, banana flour, or plantain flour.
